(19) United States
(12) Patent Application Publication
(10) Pub. No.: US 2008/0275487 A1
(43) Pub. Date: Nov. 6, 2008

(54) REMOVABLE MEDICAL FILTER WITH STAND-OFF ARMS

(76) Inventor: James A. Fleming, Bethlehem, PA (US)

Correspondence Address: RONALD L. HOFER, 122 LINDBERGH LANE, MOORESVILLE, NC, 28117, US

(21) Appl. No.: 11/742,690

(22) Filed: May 1, 2007

(51) Int. Cl. A61M 29/00

(52) U.S. Cl. 606/200

(57) ABSTRACT

A medical filter for placement in a tubular body passage. The filter has a main structure with a plurality of arms extending radially outwardly from the main structure to stand off the main structure from the wall of the tubular body passage. The filter is removed by first rotating the filter about its longitudinal axis to remove the stand-off arms from any endothelium attachments.

### BACKGROUND AND SUMMARY OF THE INVENTION

The present invention relates to a medical filter which can be placed inside a blood vessel or other body passage and to a method for retrieval of a medical filter. More specifically, the present invention relates to a medical filter having standoff arms and to its method of retrieval using a twisting technique.

Medical filters, including vena cava filters, are emplaced inside blood vessels or other body passages for the purpose of intercepting thrombus or particles while allowing free flow of blood in the vessels or other body fluid in the body passages. Medical filters often are emplaced and never retrieved, remaining effective during their time in place, and remaining permanently in place. However, while a medical filter may be implanted permanently, it may be desirable to retrieve the filter. If retrieval is desired, it should be retrieved as easily as possible with minimal trauma to the vessel or body passage. If the filter has just been implanted, retrieval may be relatively easy. But in some cases it may be desirable to remove or retrieve a medical filter after it has been in place for an extended period of time, for example, after it has been in place for more than two weeks.

Generally speaking, it is known to use catheters to emplace or deliver medical filters in a blood vessel as well as to remove medical filters following their implantation although retrieval of medical filters which have been in place for an extended period of time can be challenging. Medical filters commonly have a network of interconnected elements which extend generally radially with respect to an associated blood vessel. The filter expands radially to a size matching the anatomy of the associated blood vessel to anchor itself in the vessel to resist migration therein and, hence, part of the filter comes into contact with the wall of the blood vessel. This contacting relationship leads to endothelialization or in-growth of the wall around structural members of the filter which makes it challenging to remove the filter without trauma to the wall of the vessel.

In light of the challenge posed by removal of an extended duration medical filter, it would be beneficial to have an improved device and method for removal or retrieval of vascular and other medical filters. It would also be desirable to have a practical and economical filter and an atraumatic method for removal of the medical filter after implantation. Furthermore, it would be desirable to have a medical filter which can be implanted and removed from either a femoral or jugular approach and which can be adapted for use with a wide variety of specific medical filter designs.

Accordingly, the present invention provides an improved, removable, radially compressible and expandable medical filter for placement in a tubular body passage such as a blood vessel. In radially expanded form, the medical filter of the present invention has a main structure with a plurality of arms extending radially outwardly from the main structure. When in place in a tubular passage, the arms stand-off the main structure from the wall of the tubular body passage and anchor the main structure in the tubular passage to prevent migration of the filter in the passage. A preferred embodiment of a filter of the present invention has:

in a radially compressed state,
a main structure having a tubular shape with a longitudinal axis and first and second integral end pieces at opposite longitudinal ends of said structure; a plurality of longitudinally extending elements extending between the end pieces in a direction substantially parallel to the longitudinal axis of the medical filter, the elements having central segments; and
a plurality of arms attached to said central segments of said elements; and
in a radially expanded state, said elements being expanded radially outwardly with said arms extending radially outwardly beyond each associated central segment of said elements.

The method of the present invention involves retrieving a medical filter from a tubular body passage where the filter has a main structure and a plurality of stand-off arms attached to the main structure and standing off the main structure from the tubular body passage. The filter is retrieved by rotating the filter about its longitudinal axis to remove the stand-off arms from any associated tissue of the body passage and then withdrawing the filter into a lumen of a retrieval catheter.

Further understanding of the present invention will be had from the following description taken in conjunction with the accompanying drawings and appended claims.

### BRIEF DESCRIPTION OF THE DRAWINGS

FIG. 1 is a side elevation view, partially in section, showing a preferred embodiment of a medical filter of the present invention in the lumen of a delivery catherter;

FIG. 2 is a side elevation of the preferred embodiment of a medical filter shown in FIG. 1 in radially expanded form;

FIG. 3 is an end elevation of the medical filter of FIG. 1 in radially expanded form;

FIG. 4 is a cross-sectional view of the medical filter of FIG. 1 in operative association with a blood vessel;

FIG. 5 is an expanded view of the portion of FIG. 4 indicated by circle 5 therein;

FIG. 6 is a side elevation view, partially in longitudinal section and broken away, illustrating an initial step of a preferred embodiment of a method of retrieval of the present invention; and

FIG. 7 is a side elevation view, partially in section and broken away, showing a further step of a preferred embodiment of a method of retrieval of the present invention.

### DETAILED DESCRIPTION OF THE INVENTION

The following description of the preferred embodiments of the present invention is intended to be merely illustrative in nature, and as such, is not intended to limit in any way the present invention, its application, or uses. Numerous modifications may be made by those skilled in the art without departing from the true spirit and scope of the invention. For example, the present invention may be used in blood vessels but is also useful in other body passages.

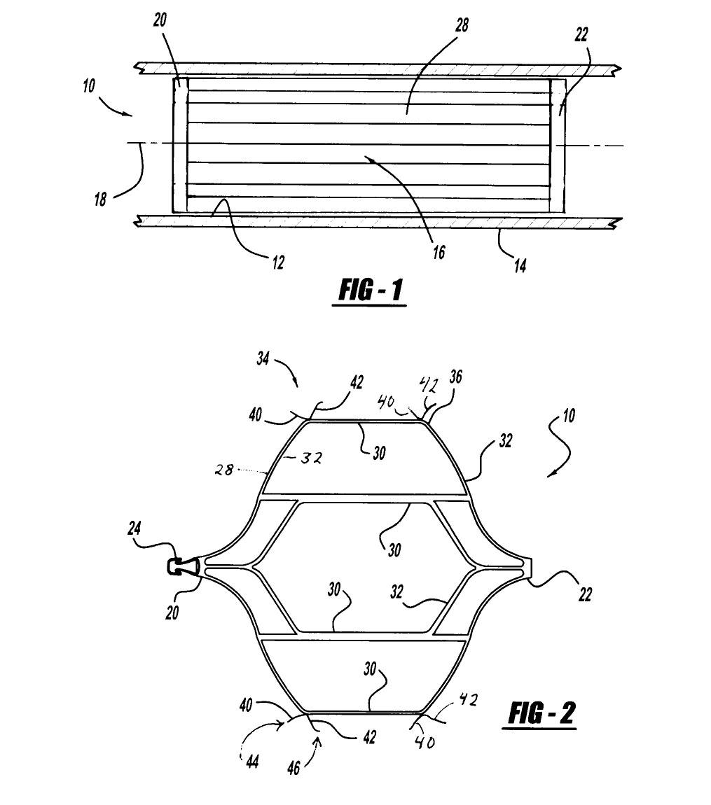

Now referring to FIG. 1, a preferred embodiment of a medical filter of the present invention is shown and indicated generally by the numeral 10. FIG. 1 shows medical filter 10 in compressed form inside lumen 12 of delivery catheter 14. Medical filter 10 has a main structure 16 having a tubular shape with a longitudinal axis 18 and first and second integral end members 20 and 22, respectively located at opposite longitudinal ends of main structure 16. As best shown in FIG. 2, end member 20 is provided with a retrieval hook 24.

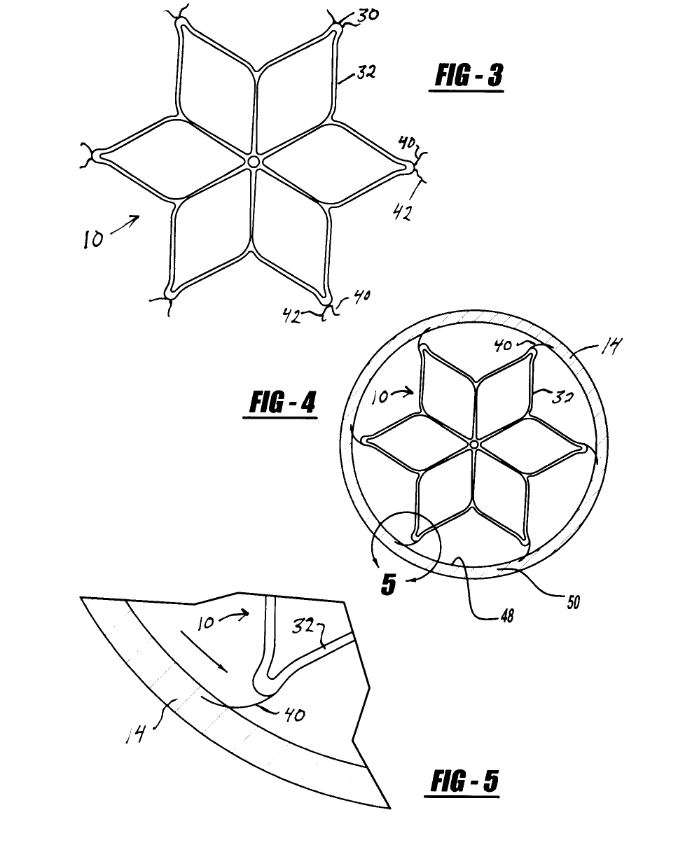

In compressed form as shown in FIG. 1 a plurality of longitudinally extending elements 28 extend between end pieces 20 and 22 in a direction substantially parallel to longitudinal axis 18 of main structure 16 and medical filter 10. As best shown in FIGS. 2 and 3, longitudinal elements 28 have central segments 30 supported by struts 32 and, in radially expanded form as shown in FIGS. 2 and 3, central segments 30 extend generally parallel to longitudinal axis 18. Struts 32 extend radially outwardly and axially from end members 20 and 22 to support central segments 30 thereon. Each central segment 30 has opposite ends 34 and 36, each of which is connected to, or formed in one piece with, a pair of struts 32. It will be appreciated by those skilled in the art that the structure of the main structure 16 described above is well known in the art and is generally disclosed, for example, in U.S. Pat. No. 6,989,021B1 the disclosure of which is specifically incorporated by reference herein. Thus, it will be appreciated that central segments 30, struts 32 and end members 20 and 22 can be separate pieces fastened together or can be integrally formed as by cutting from a single tube.

Secured to main structure 16 at each end 34 and 36 of each central segment 30 is a pair of stand-off arms 40 and 42. Stand-off arms 40 extend radially outwardly and axially in a proximate direction while stand-off arms 42 extend radially outwardly and axially in a distal direction. Preferably each stand-off arm 40 and 42 is somewhat helically shaped. Stand off arms 40 and 42 can be made from one-piece with central segments 30 or can be made separately from segments 30 and then attached thereto by any suitable means. It is contemplated that the radially distant ends 44 and 46 of stand-off arms 40 and 42 respectively will come into contact with the interior wall 48 of tubular body passage such as blood vessel 50 as best shown in FIGS. 4 and 5. Arms 40 and 42 preferably radiate outwardly from main structure 16 in a spiral pattern. As shown in the preferred embodiment, arms 40 and 42 are shown in pairs, however, it will be appreciated by those skilled in the art that arms 40 and 42 may be attached one, two or more than two, to each central segment 30 and may be attached at ends 34 and 36 or therebetween along central segment 30. It is preferred, but may not be necessary in some instances, that some arms extend radially and axially in a proximate direction while other arms extend radially and axially in a distal direction to provide anchoring against an associated vessel wall in both axial directions. The arms act as pilings to prevent the main structure from coming in contact with the vessel wall.

Each arm 40 and 42 has a free end 44 and 46 respectively so that it can be easily removed from an associated vessel wall if pulled in a direction generally opposite to the free end. Preferably, arms 40 and 42 have no mechanical interference features such as hooks, bends or corners, between the arms and the endothelium so that when the main structure 16 is twisted, i.e., rotated about its longitudinal axis 18, arms 40 and 42 will encounter little resistance other than frictional forces between the surface of the arms and the endothelium. Such frictional forces can be reduced by surface finishing, surface modification or secondary coatings applied to the arms. However, such forces are, in any event, much weaker than forces that would be encountered by mechanical interaction of a mechanical interference feature and the endothelium. On the other hand, the arms provide good resistance to movement from any fluid forces generated in the axial direction as are normally encountered in use by devices of this type.

Medical filter 10 is made of a resilient material which can be compressed to a smaller diameter and cylindrical form as shown in FIG. 1 for insertion into the lumen of a delivery catheter for delivery to a blood vessel or other tubular body passage or retrieval therefrom. In use, medical filter 10 tends to expand radially to the form illustrated in FIGS. 2 and 3 and expands radially in a blood vessel or other tubular body passage as shown in FIG. 4.

Filter 10 can be made of any suitable material using a variety of methods. Suitable conventional materials and methods are well known in the art. Nitinol and stainless steel are examples of suitable materials but other materials may be used so long as the material has the desired characteristics of strength, resilience, flexibility, biocompatibility and endurance and is suitable for the particular manufacturing technique employed. It is, of course, required that the material employed be capable of expanding to the desired shape upon ejection from the delivery catheter and reduction in diameter when withdrawn into the lumen of the retrieval catheter. Thus, the material must also be sufficiently resilient to accomplish the compression and expansion required for use of filter 10.

Suitable methods of manufacture of filter 10 include cutting a pattern into a tube to enable expansion of the tube into the desired main structure and optionally arms as well. Another suitable method is forming the main structure and arms from separate strips or wires and then joining the respective parts together by suitable methods which are well known in the art.

Of course, it will be appreciated by those skilled in the art that the main structure can have many different designs and may be modified within the spirit and scope of the present invention. For example, the main structure can have additional struts and can be symmetrical or asymmetrical in form.

Having described a medical filter of the present invention, further understanding of the unique character and advantages of the present invention will be had by an understanding of the method for its retrieval. It will be further appreciated that the present method relates to a method for retrieval of a medical filter which has been previously positioned within a blood vessel or body passage. For example and not by way of limitation, filter 10 is intended to be initially deployed in the lumen of a delivery catheter as is conventional in the art. Insertion of filter 10 into a delivery catheter can be by any conventional method including by simply pushing filter 10 into the lumen at the distal end of a delivery catheter. Alternatively, filter 10 may be inserted into the proximal end of a delivery catheter and pushed by means of a push wire or the like to the distal end of the catheter. Emplacement of filter 10 in a blood vessel or other body passage may also be done by conventional means well known in the art. For example, a delivery catheter carrying a radially compressed filter 10 may be inserted along a body vessel in a patient until its distal end is near the desired site for treatment. The delivery catheter may be inserted using either a femoral approach or a jugular approach. A push wire may be used to eject filter 10 from the lumen of the delivery catheter by pushing filter 10. As filter 10 is ejected, main structure 16 expands radially outwardly until arms 40 and 42 contact the wall of the vessel to stand main structure 16 off from the wall.

Once emplaced in a body passage such as a blood vessel, in many cases there will be no desire to retrieve filter 10 from the vessel. However, in some cases there will be a desire to retrieve filter 10 from the vessel and in such cases the present invention provides a filter 10 which facilitates retrieval. Now referring to FIGS. 4-7, further understanding can be had of the method of the present invention.

FIG. 7 illustrates the initial step of the method of retrieving filter 10 in accordance with this invention. Filter 10 is shown in place in vessel 50. Retrieval wire 60 with hook 62 is shown having been advanced through vessel 50 to a location proximate to filter 10. Hook 62 is shown having captured retrieval loop 24. As will be appreciated by the skilled artisan, such advancement may be from either a femoral or jugular approach and can be made from either direction toward retrieval loop 24, even through main structure 16. Loop 62 is preferably withdrawn into lumen 64 of retrieval catheter 66 during advancement of catheter 66 through vessel 50 to facilitate the advancement of the catheter through the vessel.

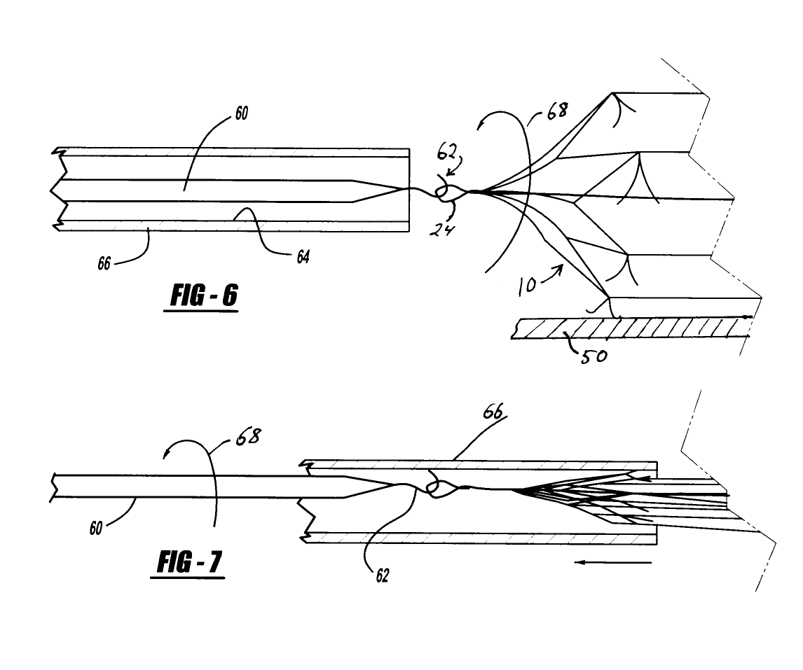

After snaring retrieval hook 24 as shown in FIG. 7 loop 62 is twisted, i.e., rotated about its longitudinal axis as indicated by arrow 68 to thereby rotate filter 10 about its longitudinal axis to withdraw the free ends of arms 40 and 42 from any endothelium from wall 48 of blood vessel 50 as is illustrated in FIGS. 4 and 5. Filter 10 can then be pulled into lumen 64 of retrieval catheter 60 as shown in FIG. 6. Alternatively, a catheter can be extended through the main structure 16 of filter 10 and then rotated to twist main structure 16 and thereby remove the free ends of arms 40 and 42 from wall 48. Then a snare can be used to capture retrieval hook 24 to then withdraw filter 10 into a suitable retrieval catheter.

While preferred embodiments of the present invention have been specifically described above, it will be appreciated by those skilled in the art that the present invention is subject to variations and modifications. For example, the filter may be cut from a single tube and have end rings which are integral, i.e., one piece, with the spine and struts. The filter may have wire struts and end rings which are tubular.

What is claimed is:

1. A removable, radially compressible and expandable medical filter for placement in a tubular body passage such as a blood vessel, the medical filter having a main structure having a snaring device for applying axial and rotational force on said main structure, said filter further having a plurality of arms extending radially outwardly from the main structure to stand off the main structure from the wall of the tubular body passage.

2. A medical filter as set forth in claim 1, wherein said arms extend both radially outwardly and axially when said filter is in a radially expanded state.

3. A medical filter as set forth in claim 2, wherein said main structure has a plurality of central segments radially spaced about a longitudinal axis of said main structure, said central segments extending parallel to said longitudinal axis, and each of said arms extending radial outwardly from one of said central segments.

4. A medical filter as set forth in claim 3, wherein each of said central segments has opposite ends and each of said arms extends from one of said opposite ends.

5. A medical filter as set forth in claim 4, wherein a pair of arms extends from each of said opposite ends with one of said pair extending axially in approximate direction and one of said ends extending axially in a distal direction.

6. A removable, radially compressible and expandable medical filter for placement in a tubular body passage such as a blood vessel, the medical filter having
in a radially compressed state,
a main structure having a tubular shape with a longitudinal axis and first and second end members at opposite longitudinal ends of said structure; a
plurality of longitudinally extending elements extending between the end pieces in a direction substantially parallel to the longitudinal axis of the medical filter, the elements having central segments; at least one of said end members carrying a retrieval hook; and
a plurality of arms attached to said central segments of said elements; and
in a radially expanded state, said elements being expanded radially outwardly with said arms extending radially outwardly beyond each associated central segment of said elements.
A medical filter as set forth in claim 1, wherein said arms extend both radially outwardly and axially when said filter is in a radially expanded state.

7. A medical filter as set forth in claim 6, wherein said main structure has a plurality of central segments radially spaced about a longitudinal axis of said main structure, said central segments extending parallel to said longitudinal axis, and each of said arms extending radial outwardly from one of said central segments.

8. A medical filter as set forth in claim 7, wherein each of said central segments has opposite ends and each of said arms extends from one of said opposite ends.

9. A medical filter as set forth in claim 8, wherein a pair of arms extends from each of said opposite ends with one of said pair extending axially in approximate direction and one of said ends extending axially in a distal direction.

10. A method of retrieving a medical filter from a body passage, said filter having a main structure and a plurality of stand-off arms attached to the main structure, said method comprising rotating the filter about its longitudinal axis to remove the stand-off arms from any endothelium attachments by said body passage.

11. A method of retrieving a medical filter as set forth in claim 10 wherein, said main structure of said medical filter has a plurality of longitudinally extending elements each having at least one stand-off arm extending radially outwardly therefrom, said method comprising the steps of rotating said filter about said longitudinal axis to thereby remove said legs from associated tissue and then withdrawing said filter into a lumen of a retrieval catheter.

12. A method of retrieving a medical filter as set forth in claim 11 wherein each said arm extends both radially outwardly and axially from said element.

13. A method of retrieving a medical filter as set forth in claim 11, wherein said rotating step is carried out by rotating a snare attached to a hook of said main structure.

14. A method of retrieving a medical filter as set forth in claim 11, wherein said rotating step is carried out by rotating said main structure by rotating a catheter freely extending longitudinally through said main structure and in rotational interfering relationship therewith

15. A method of retrieving a medical filter as set forth in claim 11, wherein each said element has at least one pair of arms extending therefrom.

16. A method of retrieving a medical filter as set forth in claim 11, wherein said body passage is a blood vessel.

17. A method of retrieving a medical filter as set forth in claim 11, wherein a plurality of said arms extend axially in a distal direction and a plurality of said arms extend axially in a proximal direction.
